(19) United States
(12) Patent Application Publication
(10) Pub. No.: US 2006/0057223 A1
(43) Pub. Date: Mar. 16, 2006

(54) Intradiscal production of autologous interleukin antagonist

(76) Inventors: Thomas M. DiMauro, Southboro, MA (US); Mohamed Attawia, Canton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/939,268

(22) Filed: Sep. 10, 2004

(51) Int. Cl. A61K 35/14

(52) U.S. Cl. 424/529

(57) ABSTRACT

Administering a buffy coat and immunoglobulin into a disc to induce the production of interleukin receptor antagonist protein IRAP within the disc is disclosed.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities. These nucleus pulposus cells may also secrete small amounts of cytokines such as IL-1β as well as matrix metalloproteinases (MMPs). These cytokines and MMPs help regulate the metabolism of the nucleus pulposus cells.

In some instances of disc degeneration disease (DDD), gradual degeneration of the intervertebral disc is caused by mechanical instabilities in other portions of the spine. In these instances, increased loads and pressures on the nucleus pulposus cause the cells to emit larger than normal amounts of the above-mentioned cytokines. In other instances of DDD, genetic factors, such as programmed cell death, or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins.

As DDD progresses, the toxic levels of the cytokines present in the nucleus pulposus begin to degrade the extracellular matrix. In particular, the MMPs (under mediation by the cytokines) begin cleaving the water-retaining portions of the proteoglycans, thereby reducing their water-retaining capabilities. This degradation leads to a less flexible nucleus pulposus, and so changes the load pattern within the disc, thereby possibly causing delamination of the annulus fibrosus. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, ejecting the nucleus pulposus from the disc and causing it to contact a local nerve root and produce sciatic pain.

Interleukin receptor antagonist protein (IRAP) is a naturally-occurring, anti-inflammatory protein produced by monocytes and neutrophils. It has been reported IRAP competitively binds to the receptor of interleukin 1β, thereby preventing IL-1β from mediating inflammation. Accordingly, it has been proposed by several investigators that IRAP be used to stop inflammation.

Some investigators have proposed treating DDD by administering biologics, such as recombinant IRAP, that specifically antagonize pro-inflammatory cytokines. For example, Maeda et al. Spine 25(2): 166-169 (2000) reports on the in vitro response to recombinant interleukin-1 receptor antagonist protein (IRAP) of rabbit annulus fibrosus exposed to IL-1. Maeda suggests that IRAP administration to the disc could be useful in inhibiting the degradation of the disc. Although Maeda reported that 100 ng rIRAP/ml appears to successfully antagonize 1 ng IL-1β/ml, recombinant IRAP is expensive.

Meijer, Inflammatory Research 52 (2003) 404-407, teaches a method of producing IRAP in whole blood samples in therapeutically relevant amounts by its physico-chemical induction in a syringe. Meijer further reports producing up to 8 ng IRAP/ml blood by such methods and states that a minimum IL-1/IRAP ratio of 1:10 is required to inhibit IL-1 activity.

U.S. Pat. No. 6,623,472 (“Reinecke I”) teaches the production of autologous IRAP by contacting blood with a syringe having an inner structure coated with immunoglobulin G. Reinecke I teaches that the IRAP produced by this method may be injected into an intervertebral disc to treat neurologically-caused back complaints. However, Reinecke I teaches that this method requires waiting at least 12-72 hours for the viable cells to incubate in the syringe in order to produce IRAP.

U.S. Pat. No. 6,713,246 (“Reinecke II”) teaches the production of autologous IRAP by contacting blood with a syringe having an etched inner barrel, and also that the IRAP may be injected into an intervertebral disc. However, Reinecke II teaches that this method requires waiting at least 12 hours (and preferably 24 hours) for the viable cells to incubate in the syringe to produce IRAP.

Although the autologously produced IRAP taught by Reinecke II avoids the expensive recombinant technology, the 12-72 hour time span involved in its production may appear excessive to the clinician and patient.

U.S. Pat. No. 5,833,984 teaches the local administration of IgA compositions having less than 20% IgG to treat inflammation.

Koch, Cytokine, 10(9), September 1998, pp. 703-5, studied the spontaneous secretion of IRAP from a degenerating intervertebral disc in vitro, and reported a secretion rate of 4.29 μg IL-1β/10⁶ cells/48 hr. Koch further suggested that therapeutic benefit of exogeneous IRAP needs to be reconsidered.

In sum, conventional treatment methodologies for administering IRAP to a disc require either expensive recombinant technologies that are subject to intense regulatory review, or excessive preparation times and efforts.

### SUMMARY OF THE INVENTION

The present inventors have developed a method of treating inflammation wherein viable cells and an immunoglobulin are both injected into a joint, whereby the immunoglobulin induces in vivo the viable cells to produce IRAP.

This method is advantageous in that sufficient in vivo production of IRAP is insured by the clinician's ability to provide large amounts of viable cells into the joint. Moreover, since the procedure can be carried out immediately after the viable cells and immunoglobulin are mixed, there is no need to wait 12-24 hours for induction to occur.

Therefore, in accordance with the present invention, there is provided a method of administering IRAP to a patient, comprising:
a) obtaining from the patient a physiologic fluid comprising viable cells,
b) mixing an IRAP-inducing composition comprising an immunoglobulin with at least a portion of the physiologic fluid thereby producing an Ig-rich fluid, and
c) administering an effective amount of the Ig-rich fluid to a location in the patient to induce the viable cells therein to in vivo produce IRAP at the location.

The present inventors further believe that simply injecting an inducing composition comprising immunoglobulin into an inflamed disc may also alleviate inflammation, as the inducer would act upon local cells (such as invading macrophages, or local nucleus pulposus cells or annulus fibrosus cells) and induce IRAP from those cells.

Accordingly, also in accordance with the present invention, there is provided a method of treating degenerative disc disease in an intervertebral disc having a nucleus pulposus and an annulus fibrosus, comprising the steps of:
a) intradiscally administering an effective amount of a formulation comprising an IRAP-inducing composition comprising an immunoglobulin into the intervertebral disc.

The present inventors further believe that simply injecting an inducing composition comprising immunoglobulin in the vicinity of an inflamed nerve root may also alleviate inflammation and treat sciatica. It is believed that the inducer will act upon the macrophages participating in the immune response to the extruded nucleus pulposus and induce those macrophages to produce IRAP.

Accordingly, also in accordance with the present invention, there is provided a method of treating sciatica, comprising the steps of:
a) epidurally administering an effective amount of a formulation comprising an IRAP-inducing composition comprising an immunoglobulin in the vicinity of a nerve root.

### DESCRIPTION OF THE FIGURES

FIG. 1 is a cross-section of a centrifugation container filled with whole blood.

FIG. 2 is a cross-section of a centrifugation container filled with centrifuged blood.

FIG. 3 is a side view of a syringe filled with lyophilized immunoglobulin having a needle inserted into the container of FIG. 2.

FIG. 4 is a side view of the syringe of FIG. 3 now filled with an immunoglobulin-buffy coat mixture.

FIG. 5 is a cross-section of a syringe of the present invention injecting an immunoglobulin- buffy coat mixture into the nucleus pulposus of an intervertebral disc.

### DETAILED DESCRIPTION OF THE INVENTION

The immunoglobulin used in the present invention must induce the production of IRAP in viable cells. Preferably, the immunoglobulin of the present invention has the quality that it not only induces the production of IRAP in human monocytes or neutrophils, it also does so without upregulating at least one of and preferably both TNF-α and IL-1β. Preferably, it also downregulates both TNF-α and IL-1β. This has the advantageous effect of insuring the inventive process produces not only favorably high amounts of anti-inflammatory molecules, but also favorably low amounts of pro-inflammatory molecules.

Preferably, the inducing composition comprises an immuglobulin selected from the group consisting of IgA, IgG, and mixtures thereof.

In another aspect of the present invention, the inventors have identified IgA as preferred immunoglobulin. Although Reinecke I teaches the use of IgG as the preferred immunoglobulin and Ruiz de Souza, Eur. J. Immunol., 1995, May:25(5): 1267-73 reports that adminstration of IgG to cultures of purified monocytes induced a dose-dependent secretion of IRAP and IL-8 without stimulating production of IL-1, TNF-α or IL-6, the remainer of the literature has not been consistent with these reports. In particular, although the literature teaches that IgG induces IRAP production in human monocytes (Arend, Immunol. Rev. 1994 June, 139, 71-8 and Andersen, Autoimmunity 1995, 22(2): 127-133), the literature appears to be somewhat equivocal about the anti-inflammatory properties of IgG. In particular, Aukrust, Blood, 84(7), Oct. 1, 1994, pp. 2136-43, reports a 3-fold increase in TNF-α level in plasma after a 3-hour exposure to IgG. Bagge, Scand. J. Rheumatol., reported that local administration of IgG had no effect upon patients with rheumatoid arthritis. Bagge, Scand. J. Rheumatol., 1996, 25(3):174-6 reports that IVIg had no effect upon inflammation in a knee joint. U.S. Pat. No. 5,833,984 (“Eibl”) reports that, “IgG appears to actually enhance inflammatory activity, which is undesirable”. See Eibl at col. 6, lines 31-32.

Accordingly, in preferred embodiments, the inducing composition comprises IgA. The literature has reported that IgA is a very promising anti-inflammatory immunoglobulin. The literature has reported that IgA not only induces the production of IRAP in monocytes (Wolf I, Clin. Exp. Immunology, 1996, 105:537-543), it also reported that IRAP also downregulates both TNF-α and IL-6 (Wolf II, Blood, 83(5) (March 1), 1994, pp. 1278-88). Moreover, Wolf I reports that the induction of human monocytes by IgA raised the IRAP level in the culture from <1 ng/ml to over 65 ng IRAP/ml in 24 hours.

Therefore, it appears that IgA can induce the production of physiologically significant amounts of autologous IRAP in less than 24 hours.

Because IgA appears to be so strongly anti-inflammatory, preferably, the inducing composition comprises at least 50% IgA, and more preferably at least 70% IgA, as measured against the sum of all immunoglobulin in the composition.

Preferably, the inducing agent is supplied from an exogenous source. In this way, the inducer can be provided to the clinician along with an appropriate delivery device.

In some embodiments, the inducer can be provided to the clinician as a lyophilized powder capable of reconstitution. In preferred embodiments, the powder can be provided within a syringe. Preferably, this syringe can also be used to draw a solution comprising viable cells from a centrifuge, and thereby mix the solution with the lyophilized powder and reconstitute the powder at the same time.

In some embodiments, the inducer can be present as a solubilizable coating upon the walls of the delivery device (into which viable cells may be drawn).

In some embodiments, the inducer can be present as a solubilizable coating upon beads housed within the delivery device (into which viable cells may be drawn).

When IgA is selected as the inducer, it is preferably delivered in a concentration range of at least 1 mg/ml, preferably at least 4 mg/ml, more preferably at least 10 mg/ml. Accordingly, if a delivery volume of 1 cc is selected, and the nucleus pulposus into which the IgA is injected is about 3 cc, then the effective IgA concentration in the nucleus pulposus should be at least about 0.25 mg/ml, more preferably at least 1 mg/ml.

In preferred embodiments, a physiologic fluid containing viable cells is obtained from the patient. Preferably, the physiologic fluid is whole blood. Whole blood contains monocytes and neutrophils capable of producing autologous IRAP and is easily obtainable from the patient. More preferably, the obtained whole blood is then fractionated by a conventional procedure (such as centrifugation or filtration) to obtain a selected portion of whole blood.

In some embodiments, the selected portion comprises the buffy coat fraction of whole blood. The buffy coat typically comprises about 5-10 vol % of whole blood Utilization of the buffy coat in the present invention is desirable because it contains a concentrated amount of monocytes and neutrophils capable of producing autologous IRAP. Typically, the cellular concentration in the buffy coat will be on the order of 10-20 fold over whole blood. In some embodiments, a fraction of the buffy coat may be used.

In other embodiments, the buffy coat is combined with other portions of blood. In some embodiments thereof, the buffy coat is combined with at least a portion of the plasma fraction. The plasma fraction contains fibrinogen and so may be useful for clotting the inducing composition to insure that the viable cells and IgA remain in the disc space or nerve root area.

In other, the buffy coat is combined with thrombin in order to produce clotting.

In some embodiments, the buffy coat is combined with at least a portion of the platelet fraction of the blood. The platelet fraction contains growth factors, such as TGF-β, which, upon release, can help stimulate extra cellular matrix production by natural disc cells.

Preferably, white blood cells are selected as the viable cells are the present invention. Because these cells are easily obtained in a concentrated form from the simple centrifugation of a small amount of blood taken from the patient. More preferably, the monocyte fraction of white blood cells is selected as the viable cells of the present invention, as monocytes have been shown to produce IRAP upon induction by immunoglobulin. In other embodiments, the white blood cell fraction is neutrophils.

In other embodiments, the viable cells may be selected from the group consisting of chondocytes, fibroblasts, nucleus pulposus cells and annulus fibrosus cells.

Preferably, the mixing container used to mix the inducer and viable cells is adapted to provide homogeneous mixing of the inducer and viable cells. In some embodiments, the container is also a delivery device, and is preferably a syringe. In other embodiments, the container is a column having a stopcock.

As noted above, in some embodiments, the inducing agent is provided as a coating upon a substrate. In some embodiments, the substrate can be an inner wall of a syringe or column. In others, the substrate may be in the forms of beads, such as glass or hydroxyapatite beads. In others, the substrate is organic and may be selected from agarose, hyaluronic acid and cellulose acetate.

Because the patient serves as the incubation receptacle for the immunoglobin-buffy coat mixture, there is no need to wait for ex vivo production of IRAP. Accordingly, in preferred embodiments, the immunoglobin-buffy coat mixture is injected into the disc less than 10 hours after the mixing step, more preferably less than 1 hour, more preferably less than one-half hour.

Upon administration into a joint space, the induction of the viable cells by immunoglobulin preferably produces an in vivo IRAP concentration of at least 10 ng IRAP/ml, more preferably at least 25 ng IRAP/ml, more preferably at least 50 ng IRAP/ml.

As the injection location is typically inflamed and has an elevated local concentration of IL-1β, the induction preferably produces a local in vivo IRAP:IL-1β ratio of at least 1000:1, more preferably at least 10,000:1. In these ratios, the IRAP will be present in amounts effective to antagonize IL-1β.

Preferably, the immunoglobin-viable cell mixture produced in the present invention is injected into an inflamed joint within the patient in a therapeutically effective amount. In some embodiments, the joint is a hip joint. In others, it is a knee joint. In others, it is an intervertberal disc. When the immunoglobin-viable cell mixture is injected into an intervertebral disc, it is either injected into the nucleus pulposus, the annulus fibrosus, or both, in order to treat low back pain. In other embodiments, the immunoglobin-viable cell mixture is injected epidurally near a nerve root in the vicinity of a ruptured intervertebral disc in order to treat sciatica.

In some embodiments, the IRAP is produced in an amount effective to reduce or eliminate inflammation. In others, the IRAP is produced in an amount effective to reduce or eliminate pain.

### EXAMPLE I

This prophetic example describes a typical method of the present invention.

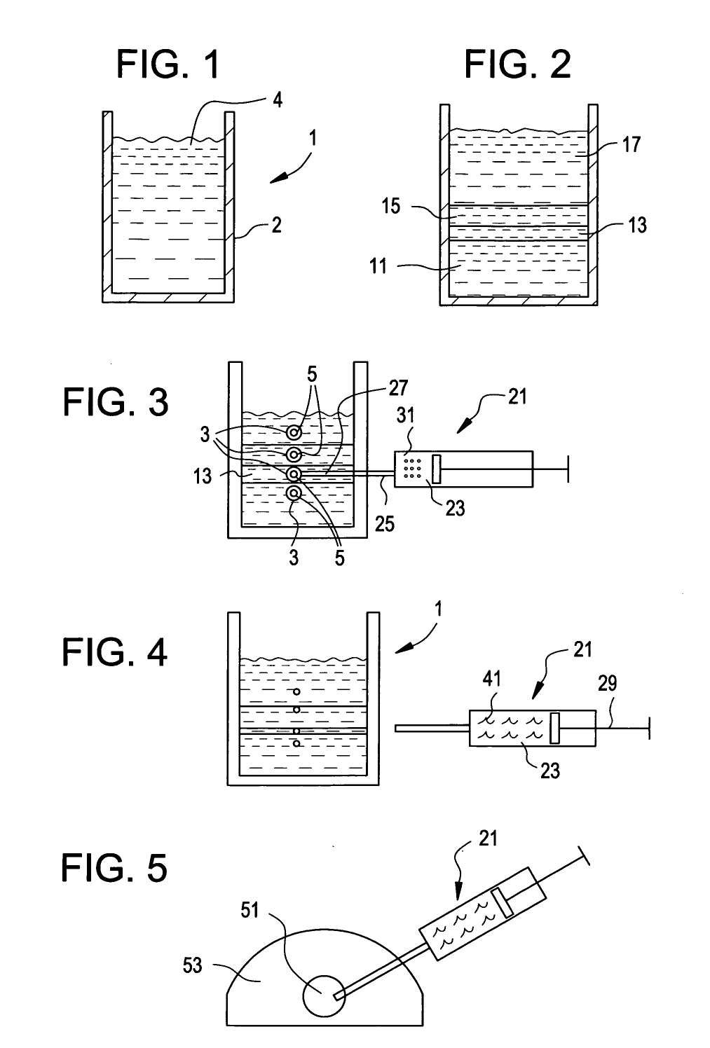

First, about 20 cc of blood is taken from the patient. Now referring to FIG. 1, the blood 4 is placed in a centrifugation container 1 adapted for centrifugation and having a side wall 2.

Now referring to FIG. 2, the blood is centrifuged to produce centrifuged blood fractions including red blood cells 11, platelets 13, buffy coat 15 and platelet poor plasma 17.

Now referring to FIG. 3, a syringe 21 having a barrel 23 containing a lyophilized immunoglobulin powder 31 and a needle 25 is provided. The centrifugation container has a plurality of side ports 3 having puncturable gaskets 5 therein. The clinician inserts the distal end 27 of the needle through the lowest gasket in the buffy coat portion 13 of the fractionated blood.

Now referring to FIG. 4, the clinician pulls back upon the plunger 29. The vacuum created by withdrawl of the plunger causes the buffy coat fluid to enter the barrel of the syringe, thereby reconstituting the immunoglobulin and producing an immunoglobulin- buffy coat mixture 41.

After reconsitution of the immunoglobulin, the clinician then waits about 5 minutes in order for the immunoglobulin to interact with the monocytes and neutrophils in the immunoglobulin-buffy coat mixture 41.

Next, the clinician uses a diagnostic test to verify that a particular disc within a patient has high levels of the particular interleukin-1β pro-inflammatory cytokine.

Next, the clinician provides a local anesthetic (such as 5 ml lidocaine) to the region dorsal of the disc of concern to reduce subcutaneous pain.

Next, the clinician punctures the skin of the patient dorsal the disc of concern with a relatively large (e.g., 18-19 gauge) needle having a stylet therein, and advances the needle through subcutaneous fat and dorsal sacrolumbar ligament and muscles to the outer edge of the intervertebral disc.

Next, the stylet is removed from the needle.

Next, the clinician receives the syringe having the inducing composition of the present invention. This syringe has a smaller gauge needle adapted to fit within the larger gauge needle. This smaller needle is typically a 22 or 24 gauge needle. The barrel of the syringe contains the formulation of the present invention.

Next, the physician advances the smaller needle co-axially through the larger needle and past the distal end of the larger needle, thereby puncturing the annulus fibrosus. The smaller needle is then further advanced into the center of the nucleus pulposus. Finally, and now referring to FIG. 5, the clincian depresses the plunger of the syringe 21, thereby injecting between about 0.5 and 1 ml of the formulation into the nucleus pulposus 51 of the intervertebral disc 53.

In one embodiment of this Example, filtration and dewatering of blood is carried out in accordance with U.S. Pat. No. 5,733,545 (Hood) to obtain a buffy coat having about 14×10⁶ monocytes/ml. Mixing 1 ml of this monocyte-rich buffy coat with lyophilized IRAP should produce about 910 ng of IRAP upon induction (based upon Wolf's production rate of 65 ng IRAP/10⁶ monocytes). Injecting 1 ml of this mixture into a 3 cc nucleus pulposus should produce about 910/4, or about 22 ng IRAP/ml.

By way of comparison, Maeda taught that a level of about 50-100 ng IRAP/ml was needed to provide a therapeutic level of IRAP to antagonize 1 ng IL-1/ml.

Also by way of comparison, since O'Neill reports a level of about 3 ng IL-1/ml in degenerating disc, and Meijer reports needing a 10/1 ratio of IRAP/IL-1, it is estimated that about 30 ng IRAP/ml would be an effective concentration in the nucleus pulposus.

We claim:

1. A method of administering IRAP to a patient, comprising:
a) obtaining from the patient a physiologic fluid comprising viable cells,
b) mixing an IRAP-inducing composition comprising an immunoglobulin with at least a portion of the physiologic fluid thereby producing an Ig-rich fluid, and
c) administering an effective amount of the Ig-rich fluid to a location in the patient to induce the viable cells therein to in vivo produce IRAP at the location.

2. The method of claim 1 wherein the portion of the physiologic fluid is a blood fraction.

3. The method of claim 1 wherein the portion of the blood fraction is a buffy coat.

4. The method of claim 1 wherein the portion of the physiologic fluid is platelet-rich plasma.

5. The method of claim 1 wherein the portion of the physiologic fluid comprises a buffy coat and platelet-poor plasma.

6. The method of claim 1 wherein the physiologic fluid is whole blood.

7. The method of claim 1 wherein the portion of the physiologic fluid comprises fibrinogen.

8. The method of claim 1 wherein the viable cells are monocytes.

9. The method of claim 1 wherein the viable cells are taken from the patient's blood.

10. The method of claim 1 wherein the viable cells are chondrocytes.

11. The method of claim 1 wherein the viable cells are neutrophils.

12. The method of claim 1 wherein the viable cells are disc cells.

13. The method of claim 1 wherein the viable cells are nucleus pulposus cells.

14. The method of claim 1 wherein the viable cells are annulus fibrosus cells.

15. The method of claim 1 wherein the viable cells are present in a concentrated form.

16. The method of claim 1 wherein the viable cells are autologous.

17. The method of claim 1 wherein the immunoglobulin is selected from the group consisting of IgA, IgG, and mixtures thereof.

18. The method of claim 1 wherein the immunoglobulin is IgA.

19. The method of claim 1 wherein the immunoglobulin is IgG.

20. The method of claim 1 wherein the IRAP-inducing composition comprises IgA and IgG.

21. The method of claim 1 wherein the IRAP-inducing composition comprises at least 50% IgA, as measured against a sum of immunoglobulin in the composition.

22. The method of claim 1 wherein the IRAP-inducing composition comprises at least 70% IgA, as measured against a sum of immunoglobulin in the composition.

23. The method of claim 1 wherein the physiologic fluid is housed within a mixing container during step b).

24. The method of claim 23 wherein the IRAP-inducing composition is a solubilizable coating upon a wall of the mixing container.

25. The method of claim 23 wherein the IRAP-inducing composition is a solubilizable powder located within the container.

26. The method of claim 23 wherein the mixing container is a syringe.

27. The method of claim 1 wherein the mixing step is carried out in less than 10 hours.

28. The method of claim 1 wherein the mixing step is carried out in less than 1 hour.

29. The method of claim 1 wherein the induction produces an in vivo IRAP concentration of at least 10 ng/ml IRAP.

30. The method of claim 1 wherein the induction produces an in vivo IRAP concentration of at least 25 ng/ml IRAP.

31. The method of claim 1 wherein the induction produces an in vivo IRAP concentration of at least 50 ng/ml IRAP.

32. The method of claim 1 wherein the location further comprises IL-10, and the induction produces an in vivo IRAP: IL-1β ratio of at least 1000:1.

33. The method of claim 1 wherein the location further comprises IL-1β, and the induction produces an in vivo IRAP: IL-1β ratio of at least 10,000:1.

34. The method of claim 1 wherein the Ig-rich fluid is injected into a joint.

35. The method of claim 1 wherein the Ig-rich fluid is injected into an intervertebral disc.

36. The method of claim 1 wherein the Ig-rich fluid is injected into a nucleus pulposus of the intervertebral disc.

37. The method of claim 1 wherein the Ig-rich fluid further comprises platelet releasate.

38. The method of claim 1 wherein the Ig-rich fluid further comprises stem cells.

39. The method of claim 1 wherein the Ig-rich fluid further comprises fibrinogen.

40. The method of claim 1 wherein the Ig-rich fluid further comprises thrombin.

41. The method of claim 1 wherein the Ig-rich fluid is substantially free of cells.

42. A method of treating degenerative disc disease in an intervertebral disc having a nucleus pulposus and an annulus fibrosus, comprising the steps of:
a) intradiscally administering an effective amount of a formulation comprising an IRAP-inducing composition comprising an immunoglobulin into the intervertebral disc.

43. A method of treating sciatica, comprising the steps of:
a) epidurally administering an effective amount of a formulation comprising an IRAP-inducing composition comprising an immunoglobulin in the vicinity of a nerve root.

44. A centrifugation container adapted for centrifuging blood comprising a side wall having at least one side port disposed therein.

45. The centrifugation container of claim 42 wherein the side port comprises a puncturable gasket adapted for receiving a needle.

46. The centrifugation container of claim 42 wherein the side wall has a plurality of side ports, each port comprising a puncturable gasket adapted for receiving a needle.

47. The centrifugation container of claim 42 wherein the plurality of side ports are vertically aligned.

48. A method of producing IRAP, comprising:
a) obtaining from the patient a physiologic fluid comprising viable cells,
b) mixing an IRAP-inducing composition comprising IgA with at least a portion of the physiologic fluid thereby producing an Ig-rich fluid.

49. The method of claim 48 further comprising the step of:
c) allowing the Ig-rich fluid to produce IRAP ex vivo, and
d) administering an effective amount of the IRAP to a location in the patient.

50. The method of claim 48 further comprising the step of:
c) administering an effective amount of the Ig-rich fluid to a location in the patient to induce the viable cells therein to in vivo produce IRAP at the location.
